(19) United States
(12) Patent Application Publication
(10) Pub. No.: US 2008/0155984 A1
(43) Pub. Date: Jul. 3, 2008

(54) Reforming system for combined cycle plant with partial CO2 capture

(76) Inventors: Ke Liu, Rancho Santa Margrita, CA (US); Michael John Bowman, Niskayuna, NY (US); Stephen Duane Sanborn, Copake, NY (US); Andrei Tristan Evulet, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/648,925

(22) Filed: Jan. 3, 2007

(51) Int. Cl. F01K 25/06; F02G 3/00; C07C 27/04; B01J 19/00

(52) U.S. Cl. 60/649; 60/783; 60/39.12; 518/704; 422/198

(57) ABSTRACT

A combined cycle system includes, a pre-steam-methane-reformer operating at a temperature of less than about 800 degrees Celsius to reform a mixed fuel stream to generate a first reformate stream, a water-gas-shift reactor to convert carbon monoxide in the first reformate stream to carbon dioxide and form a second reformate stream, a carbon dioxide removal unit for removing carbon dioxide from the second reformate stream and form a carbon dioxide stream and a third reformate stream; wherein less than about 50 percent of the carbon contained in the mixed fuel stream is recovered as carbon dioxide by the removal unit, a gas turbine unit for generating power and an exhaust stream, and a steam generator unit configured to receive the exhaust stream, wherein the heat of the exhaust stream is transferred to a water stream to generate the steam for the mixed fuel stream and for a steam turbine.

### BACKGROUND

Carbon dioxide (CO₂), one of the so-called greenhouse gases, is produced during the combustion of fossil fuels in furnaces and power plants. Recent scientific studies have shown that emissions of CO₂ and other greenhouse gases, such as methane (CH₄) and nitrogen oxides (N₂O), can have a significant effect on climate change. The prospect of climate change caused, at least in part, by emission of CO₂ and other greenhouse gases has led to international concern and to international treaties, such as the Kyoto Protocol.

Because of the national and international concern, power producers have been attempting to reduce the levels of CO₂ produced by power plants. Many newer power plants are combined cycle plants fired by natural gas, or “NGCC” plants. While these plants produce significantly less CO₂ then coal-fired power plants, difficulty remains in meeting the ever-increasing emissions standards. Recently, policy makers in Europe proposed creation of a maxima quota of CO₂ a given power plant may release per year. It has been proposed that CO₂ emissions exceeding that quota will have to pay a “carbon tax” on the excess amount. In fact, Sweden already has a carbon tax in place. Likewise, Norway, Finland, and the Netherlands have recently enacted carbon taxes. Similar carbon tax proposals have been discussed by California and other U.S. states determined to improve air quality standards.

Existing power plants can utilize steam methane reforming (SMR), autothermal reforming (ATR) and catalytic partial oxidation (CPO) to convert natural gas (NG) to a syngas or reformate comprising hydrogen and carbon monoxide for use in gas turbine generators, as well as hydrogen, for use in ammonia production or refineries. While the use of reformate can advantageously reduce NOₓ emissions, the reformation reactions of natural gas (NG), as well as the combustion required for power generation can create large amounts of CO₂. In order to reform all of the NG, the required reformer would need to be very large and expansive. Moreover, if a SMR reformer was used, the furnace side of the reformer would need to be operated at temperatures as high as 2,600 degrees Fahrenheit (° F.). At such temperatures, the SMR reformer would need to be made of costly high temperature alloys. Perhaps an even bigger hurdle would be capturing the large amount of CO₂ generated by such reformers. Capturing such large amounts of CO₂ would be costly and bring down the overall efficiency of the plant, as more fuel would be required to capture the excess CO₂ emissions. Large capital investments would thus be required to provide existing power plants the capability of complying with the increasingly restrictive CO₂ emissions standards.

Therefore, there is a need for a power plant, which can utilize a cheaper, lower temperature reformer to convert the NG, and capture a portion of the CO₂ there created. Such a system would reduce capital and operational costs by running at lower temperatures, using recycle streams to increase efficiency, and capturing only the CO₂ emissions generated beyond the plant's yearly quota, thereby, avoiding costly carbon taxes. Moreover, it would be advantageous if the system could be retrofitted to existing NGCC power plants.

### BRIEF DESCRIPTION

Disclosed herein are natural gas combined cycle systems with partial carbon dioxide capture and methods for operating the systems. In one embodiment, a combined cycle system includes, a reformer unit including a pre-steam-methane-reformer configured to operate at a temperature of less than about 800 degrees Celsius and to reform a mixed fuel stream to generate a first reformate stream, wherein the mixed fuel stream comprises a first fuel and a steam, a shift reaction unit comprising a water-gas-shift reactor configured to convert carbon monoxide in the first reformate stream to carbon dioxide and form a second reformate stream, a carbon dioxide removal unit configured to remove carbon dioxide from the second reformate stream and form a carbon dioxide stream and a third reformate stream; wherein less than about 50 percent of the carbon contained in the mixed fuel stream is recovered as carbon dioxide by the carbon dioxide removal unit, a gas turbine unit configured for receiving a mixture of the third reformate stream and a second fuel and generating power and an exhaust gas stream, wherein the exhaust gas stream provides heat to reform the mixed fuel stream, and a steam generator unit configured to receive the exhaust gas stream, wherein the heat of the exhaust gas stream is transferred to a water stream to generate a cooled exhaust stream and the steam for a steam turbine and the mixed fuel stream.

A method for producing power and partially capturing carbon dioxide includes, reforming a mixed fuel stream including a first fuel and a steam in a pre-steam-methane-reformer to produce a first reformate stream including hydrogen, carbon monoxide, and steam, converting the steam and the carbon monoxide in the first reformate stream to a second reformate stream comprising carbon dioxide and hydrogen in a water-gas-shift reactor, removing the carbon dioxide from the second reformate stream in a carbon dioxide removal unit to produce a carbon dioxide stream and a third reformate stream, wherein less than about 50 percent of the carbon contained in the mixed fuel stream is recovered as carbon dioxide by the carbon dioxide removal unit, combusting a mixture of the third reformate stream and a second fuel stream in a gas turbine unit to generate power and produce an exhaust stream, and utilizing heat in the exhaust stream to generate the steam in a heat recovery steam generator, wherein the steam is used to generate power and form the mixed fuel stream with the first fuel.

In another embodiment, a combined cycle system includes, a combination unit including a heat recovery steam generator, wherein the heat recovery steam generator comprises at least two stages, wherein a first stage includes a pre-steam-methane-reformer, wherein the pre-steam-methane-reformer is configured to operate at a temperature of less than about 800 degrees Celsius and utilize heat from a hot gas turbine exhaust stream to reform a mixed fuel stream to form a first reformate stream, and wherein a second stage utilizes heat from the exhaust stream to form a steam, a shift reaction unit including a water-gas-shift reactor configured to convert carbon monoxide in the first reformate stream to carbon dioxide and form a second reformate stream, a carbon dioxide removal unit configured to remove carbon dioxide from the second reformate stream and form a carbon dioxide stream and a third reformate stream; wherein less than about 50 percent of the carbon contained in the mixed fuel stream is recovered as carbon dioxide by the carbon dioxide removal unit, and a gas turbine unit configured for receiving a second fuel and the third reformate stream and generating power and the exhaust stream.

### DESCRIPTION OF THE DRAWINGS

Referring now to the figures wherein the like elements are numbered alike:

FIG. 1 illustrates an exemplary combined cycle power system with partial CO₂ capture; and

FIG. 1 illustrates another exemplary combined cycle power system with partial CO₂ capture.

### DETAILED DESCRIPTION

Combined cycle power systems and processes utilizing pre steam-methane-reformers (SMR) and partial CO₂ capture units are disclosed herein. The combined cycle system combines the Rankine (steam turbine) and Brayton (gas turbine) thermodynamic cycles by using heat recovery to capture the energy in the gas turbine exhaust gases for steam production. In contrast to prior art combined cycle plants using conventional reformers, the systems and methods disclosed herein advantageously use a low-temperature pre-SMR to reform only a portion of natural gas (NG) for capture of CO₂ emissions over a specified allowance. In conventional SMRs the reaction must occur under high temperatures, e.g., greater than 1,000 degrees Celsius (° C.) for full conversion of the methane to hydrogen. In the pre-SMR disclosed herein, however, reaction temperatures are about 550° C. to about 800° C., specifically about 600° C. to about 750° C., even more specifically 650° C. Because it is desired to capture less than about 50 percent of the carbon in the fuel stream, a conversion efficiency of less than or equal to about 70 percent methane to hydrogen and carbon monoxide is all that is required by the pre-SMR. Moreover, the disclosed systems use regenerative heat exchangers to recover the heat of reformate exiting from the pre-SMR and a water-gas-shift (WGS) reactor to pre-heat the NG and steam that is fed to the pre-SMR, thereby increasing overall system efficiency. The pre-SMR may also be retrofitted to an existing heat recovery steam generator (HRSG), thereby obtaining the advantages of the disclosed system without the added capital cost or required space of a separate SMR unit.

The terminology used herein is for the purpose of description, not limitation. Specific structural and functional details disclosed herein are not to be interpreted as limiting, but merely as a basis for the claims and a representative source for teaching one skilled in the art to variously employ the invention. Furthermore, as used herein, the terms “first”, “second”, and the like do not denote any order or importance, but rather are used to distinguish one element from another, and the terms “the”, “a”, and “an” do not denote limitation of quantity, but rather denote the presence of at least one of the referenced item. The modifier “about” used in connection with a quantity is inclusive of the stated value and has the meaning dictated by context, (e.g., includes the degree of error associated with measurement of the particular quantity). Additionally, all ranges directed to the same quantity of a given component or measurement is inclusive of the endpoints and independently combinable.

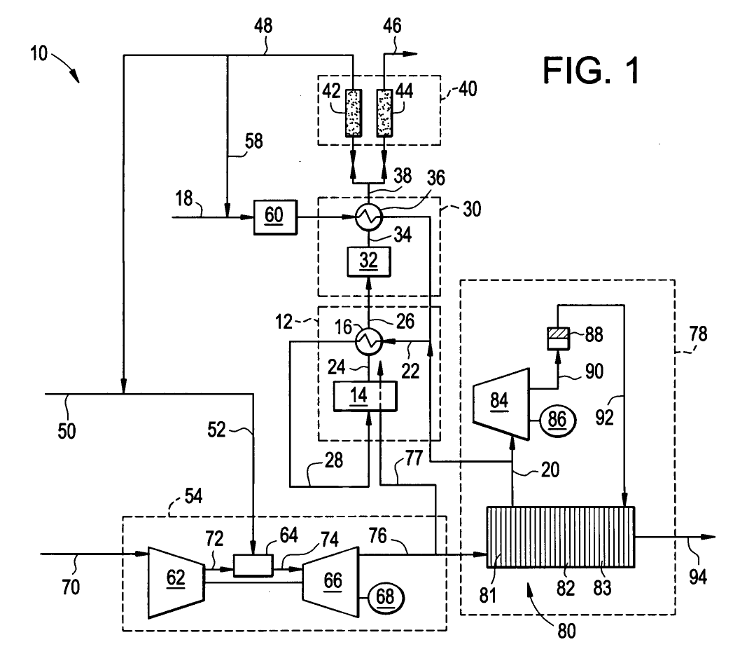

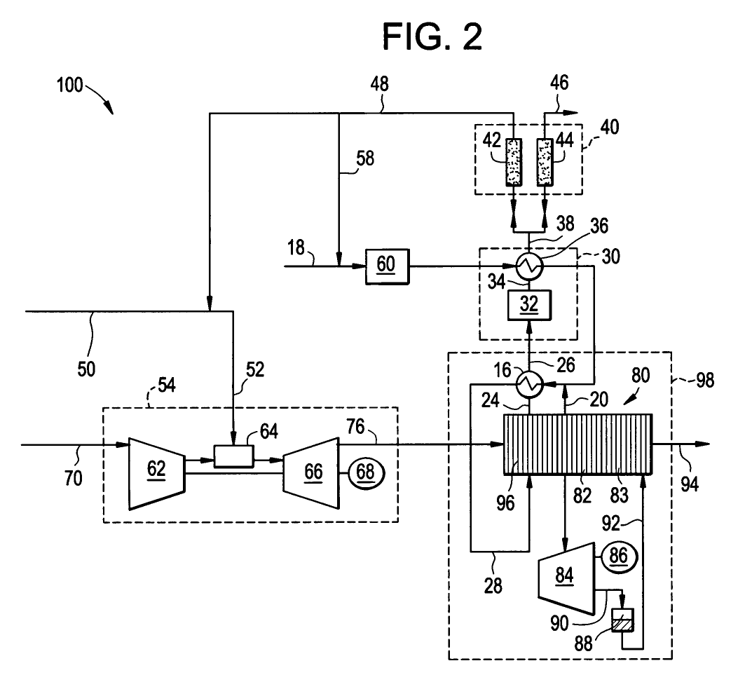

FIG. 1 represents an exemplary NGCC power system 10 for producing power and capturing CO₂ emissions. The power system 10 includes a reformer unit 12 having a pre-SMR 14 and a heat exchanger 16. The reformer unit 12 is configured to receive a first fuel 18 and a steam 20, combined as a mixed fuel stream 22, and produce a first reformate stream 24 comprising carbon monoxide, hydrogen, unconverted fuel, and steam. The heat exchanger 16 transfers the heat from the first reformate stream 24 to the mixed fuel stream 22 to generate a cooled first reformate stream 26 and a heated mixed fuel stream 28. The power system 10 further includes a shift reaction unit 30. The cooled first reformate stream 26 is sent to the shift reaction unit 30, wherein the carbon monoxide (CO) and steam in the stream 26 is converted to carbon dioxide and hydrogen in a WGS reactor 32. A second reformate stream 34 exits the WGS reactor and enters a heat exchanger 36. The heat exchanger 36 transfers the heat from the second reformate stream 34 to the first fuel 18 to generate a cooled second reformate stream 38 and a heated first fuel 18. The cooled second reformate stream 38 is sent to a CO₂ removal unit 40. The CO₂ removal unit 40 includes an amine absorber 42 and a regeneration tower 44 and is configured to remove carbon dioxide from the cooled second reformate stream 28 to form a carbon dioxide stream 46 and a third reformate stream 48 comprising hydrogen, carbon monoxide, and unconverted fuel.

The third reformate stream 48 is mixed with a second fuel 50 to form a hydrogen-enriched fuel stream 52, which is sent to a gas turbine unit 54. Optionally, a portion 58 of the third reformate stream 48 may be sent to a hydrodesulfurization (HDS) unit 60 to provide the hydrogen that is needed for HDS processing of the first fuel 18. The gas turbine unit 54 includes a compressor 62, a combustor 64, a gas turbine 66, and a generator 68. An oxidant 70 is compressed by the compressor 62 before mixing with the hydrogen-enriched fuel stream 52. The compressed oxidant 72 and the hydrogen-enriched fuel stream 52 are combusted in the combustor 64 to produce heat energy and hot compressed combustion exhaust gas mixture 74, which is sent to the gas turbine 66. The compressed combustion exhaust gas mixture 74 is expanded to drive the turbine, and is subsequently discharged as an exhaust stream 76 to the steam generator unit 78. A portion (77) of the gas turbine exhaust stream 76 is diverted to the pre-SMR 14 to provide the heat to reform the mixed fuel stream 28. Rotation of the turbine by the expanded high pressure mixed gas is converted to power by means of the generator 68 in a manner generally known to those skilled in the art.

The steam generation unit 78 includes a HRSG 80, a steam turbine 84, and a steam generator 86. The HRSG 80 has three stages 81, 82, and 83 for utilizing the waste heat from exhaust gas 76 to produce the steam 20. The steam 20 is combined with the first fuel 18 to form the mixed fuel stream 22. The steam 20 is further used to both drive the reforming reaction in the pre-SMR 14 and to produce power via the steam turbine 84 and the steam generator 86. The steam generation unit 78 may further include a condenser 88 to condense the steam turbine outlet stream 90 to form a water stream 92. The water stream 92 may be recycled to the HRSG 80 for steam generation. The cooled exhaust stream 94 may be vented to the environment.

Turning back now to the reformer unit 12, the pre-SMR 14 is configured to reform the first fuel through a conventional steam reforming process. The pre-SMR, however, reforms that fuel at temperatures lower than that of existing SMR reformers; therefore, the methane in the NG is only partially converted to syngas (comprising hydrogen and CO) as will be discussed in greater detail below. The fuel 18 may comprise any suitable gas or liquid. For ease in discussion, the first fuel 18 will be referred to as being NG. NG refers to a mixture of gases that principally includes methane together with varying quantities of ethane, propane, butane, and other gases. Typically, about 5 to about 50 percent of the NG feed to the NGCC system 10 may be fed to the pre-SMR 14. Specifically, about 10 to about 30 percent of the NG feed is converted by the pre-SMR, even more specifically about 20 percent. The main constituent of natural gas is methane (CH₄), which reacts with steam in a two-step reaction to produce hydrogen and carbon dioxide. In accordance with the present technique as shown in FIG. 1, the first reaction takes place in the pre-SMR 14, where methane reacts with steam to produce hydrogen and carbon monoxide according to the following reactions (1).

CH₄+H₂O [symbol] CO+3H₂ ΔH^{o}₂₉₈=+251 kJ mol⁻¹ (1)

The steam reforming reaction (1) is endothermic. Because of this, the steam reforming process is energy intensive and significant heat is needed in the overall reforming process. As stated previously, the pre-SMR 14, operates with reaction temperatures of about 500° C. to about 800° C., specifically about 600° C. to about 700° C., even more specifically 650° C. Because it is desired to capture less than about 50 percent of the carbon in the first fuel 18, a conversion efficiency of less than or equal to about 70 percent methane to hydrogen and carbon monoxide is all that is required by the pre-SMR 14. As such, the pre-SMR is able to advantageously operate at lower temperatures, thereby reducing operational costs, as well as capital cost through the elimination of the need for expensive high-temperature alloys. The pre-SMR 14 may comprise a number of tubes through which heat for the endothermic reaction (1) is transferred from the hot gas turbine exhaust stream to the SMR catalyst. The heated mixed fuel stream 28 is passed over a steam reforming catalyst and is converted to a first reformate stream 24 comprising a mixture of hydrogen, CO, CO₂, unconverted fuel, and steam. The now cooled portion 77 of the gas turbine exhaust stream can then be sent to stacks for preparation before venting to the atmosphere. The pre-SMR catalyst can be any conventional SMR catalyst known to those skilled in the art, such as a nickel-based catalyst. Optionally, the reformer unit 12 may further comprise a feedstock saturator circuit suitable for admixing the steam 20 to the first fuel 18.

After the first reformate stream 24 is optionally cooled by the heat exchanger 16, the cooled first reformate stream 26 enters the shift reaction unit 30. The second reaction of the steam reforming process takes place in the WGS reactor 32, where the CO and steam of the cooled first reformate stream 26 is converted to CO₂ and hydrogen according to the following reaction (2).

CO+H₂O [symbol] CO₂+H₂ ΔH=−41.16 kJ/mol (2)

The shift reaction (2) is mildly exothermic and takes place in the presence of a shift catalyst. The first reformate stream 26, therefore, increases in temperature across the catalyst beds as the reaction proceeds. The shift catalyst may include a high temperature shift catalyst (HTS) or a low temperature shift catalyst (LTS) or a combination of HTS and LTS catalysts. In the WGS reactor 32, reaction temperatures may be about 200° C. to about 600° C. Maintaining low temperatures, however, will drive reaction (2) to the right, i.e., will produce more hydrogen and CO₂ and less steam and CO. The WGS reactor, therefore, may operate in a temperature range of about 300° C. to about 400° C., more specifically about 350° C. Conversion of the first reformate stream 26 into CO₂ and hydrogen creates the second reformate stream 34. Furthermore, the reformer unit 12 and the shift reaction unit 30 may be separate pieces of apparatus (as shown in FIG. 1) or there may be a single piece of apparatus that comprises both the pre-SMR 14 and the WGS reactor 32.

The carbon dioxide removal unit 40 may comprise an amine absorber 42 and a regeneration tower 44. The second reformate stream 34 may be cooled to a suitable temperature by heat exchanger 36 to better make use of the chemical absorption of CO₂ using amines. This technique is based on alkanol amine solvents that have the ability to absorb CO₂ at relatively low temperatures, and are easily regenerated by raising the temperature of the rich solvents. The solvents used in this technique may include, for example, triethanolamine, monoethanolamine, diethanolamine, diisopropanolamine, diglycolamine, methyldiethanolamine, and the like. As stated above, the CO₂ captured may be less than about 50 percent of the carbon in the first fuel 18. Enough CO₂ is captured to avoid potential carbon tax penalties for emissions over annual CO₂ emissions quotas. However, capital investment and operational costs are reduced and energy efficiencies are increased in the disclosed system compared to prior art systems having full CO₂ capture. The CO₂ stream 46 thus produced and captured, may be readily transported to desired locations. For example, the CO₂ may be conveniently transported to locations where it can be injected into suitable underground structures for storage (sequestration), or to oilfields for Enhanced Oil Recovery (EOR), or to be used in manufacturing processes.

The remaining stream from the carbon dioxide removal unit 40 is a third reformate stream 48 mainly comprising hydrogen, CO, unutilized fuel, and water. This stream is sent to the gas turbine unit 54 for combustion. Optionally, a portion 58 of this stream may be sent to a HDS unit 60. In the HDS unit 60, sulfur contained in the first fuel 18 is converted to hydrogen sulfide by a hydrodesulfurizer in a desulfurization column of the HDS unit 60. The hydrogen sulfide is then adsorbed and removed by a sulfur absorber or an adsorption unit downstream of the desulfurization column in the HDS unit 60. It is advantageous to remove the sulfur from the first fuel 18 because the sulfur can poison the pre-steam reforming catalyst. By diverting a portion 58 of the third reformate stream 48, the hydrogen required for the HDS process is supplied in a closed-loop cycle, rather than needing a separate hydrogen source stream. The HDS unit 60 may operate at temperatures of about 200° C. to about 400° C., specifically about 250° C. to about 350° C. The catalyst used in the HDS process may be existing HDS catalyst(s), such as those commercially produced by Sud Chemie or Haldor Topsoe, e.g., sulphides of cobalt and molybdenum or nickel and molybdenum.

The third reformate stream 48 and the second fuel 50 are mixed together, prior to entering the gas turbine unit 54, to form a hydrogen-enriched fuel stream 52. The second fuel comprises the remainder of the fuel, i.e., NG, sent to the power system 10. Typically, about 50 to about 95 percent of the NG feed to the NGCC system 10 may be consumed as fuel in the gas turbine unit 54. Specifically, about 70 to about 90 percent of the NG feed is consumed by the combustor 64, even more specifically about 80 percent in order to capture about 10% of the total CO2 of the combined cycle plant. The hydrogen-enriched fuel stream 52 is injected into the combustor 64, where it is burned in the presence of a compressed oxidant 72, to produce a hot compressed combustion exhaust gas mixture 74. The hydrogen-enriched fuel expands flame stability in the combustor 64 compared to use of the second fuel 50 alone, thus the combustion can be leaner, and the flame temperature lower. The result is a combustor exhaust gas having lower NOₓ emissions due to the lower flame temperature in the combustor 64. The combustor will also have the ability to turn-down further compared to a combustor using the second fuel 50 only. Moreover, by doping natural gas with hydrogen, a greater operability window for the combustor to generate power and simultaneously maintain low emissions is created. The hot compressed mixed gas 74 then exits the combustor 64 and is passed through the gas turbine 66 where the hot compressed mixed gas 74 partially cools and expands thereby generating mechanical power. The mechanical power is converted to electrical power by the generator 68. The expanded and partially cooled exhaust gas 76 exits the gas turbine 66 and enters the steam generator unit 78.

The steam generator unit 78 includes a HRSG 80, which recovers the waste heat from the exhaust gas 76 and generates the steam 20. The HRSG 80 has three stages 81, 82, 83 for cooling the exhaust gas 76 and generating the steam 20. A portion of the steam 20 is sent to a steam turbine 84 where the steam 20 is expanded and cooled, thereby generating mechanical power. The mechanical power is subsequently converted to electrical power by the generator 86. The expanded, cooled steam exits the turbine 84 and is further cooled and condensed in a condenser 88 to form a water stream 92 that is introduced into the HRSG 80. The now cooled exhaust gas 94 is sent to stacks for venting into the atmosphere. As stated above, the remainder of the steam 20 is combined with the first fuel 18 to form the mixed fuel stream 22, which is then sent to the pre-SMR 14. By sending the remainder of the steam 20 to the pre-SMR 14 there is advantageously no need for the system 10 to have an additional steam generator in order to provide the steam required to drive the reformation reaction.

Turning now to FIG. 2, a second exemplary power system 100 is illustrated. Note that a description of components common to those in the first embodiment of FIG. 1 is omitted here.

In FIG. 2, the first stage 81 (as shown in FIG. 1) of the HRSG 80 is a pre-SMR 96. The power system 100 of FIG. 2 combines the steam generator unit 78 (of FIG. 1) with the reformer unit 12 (of FIG. 1) to form a combination unit 98. The first stage 96 of the HRSG 80 is modified to function as a pre-SMR. The HRSG 80 may be a shell-tube type heat exchanger. As such, pre-SMR catalyst may be packed into the tube side (i.e., cold side) of the first stage 96 of the HRSG 80. The exhaust gas 76 may be passed through the shell side (i.e., hot side) of the first stage 96 of the HRSG 80. The first stage 96 is configured to operate in a temperature range of about 600° C. to about 900° C. The pre-heated mixed fuel stream 28 is passed over the pre-SMR catalyst in the tubes of the first stage 96 in order to reform the fuel and generate the first reformate stream 24, as described above in the first embodiment. The hot gas turbine exhaust gas 76, flowing through the shell side of the first stage 96, supplies the heat required to drive the endothermic steam reforming reaction (1) as described above. The remaining stages 82 and 83 of the HRSG 80 transfer the remainder of the heat in the exhaust gas 76 to the water 92 in order to generate the steam 20. Optionally, the heat exchanger 16 may be included as part of the combination unit 98 for transferring heat from the first reformate stream 24 to the mixed fuel stream 22.

By modifying the first stage 96 of the HRSG 80 to be a pre-SMR, the capital cost of having the disclosed NGCC power system with partial CO₂ is reduced. The cost of building a separate pre-SMR is spared and the space required to install such a unit is saved as well. Moreover, as most power plants include HRSGs, these units can be modified to include a pre-SMR stage, thereby reducing the money and space needed to retrofit an existing power plant to obtain the same low-cost advantages of the disclosed system for partial CO₂ capture in an existing NGCC plant.

As stated above the fuel used in the disclosed systems preferably comprises NG. The systems, however, may be configured to use any suitable gas or liquid as fuel, such as for example, bio-gas (comprising mainly methane), liquefied petroleum gas (LPG), naphtha, butane, propane, diesel, kerosene, ethanol, methanol, aviation fuel, a coal derived fuel, a bio-fuel, an oxygenated hydrocarbon feedstock, and mixtures thereof. It should be noted that the first fuel 18 and the second fuel 50, each might be chosen from any of these examples of fuels described herein. In one embodiment the first fuel 18 and the second fuel 50 are the same. The oxidant 70 used in the disclosed systems may comprise any suitable gas containing oxygen, such as for example, air, oxygen-rich air, oxygen-depleted air, or oxygen from an air-separation-unit (ASU).

The NGCC power systems described herein have many advantages. By incorporating the low temperature, low cost pre-SMR unit built for partial methane conversion into the system, fuel costs, capital costs, and energy costs can be reduced compared to systems employing full SMR reformers built for full conversion of methane. Similarly, capital and energy costs are reduced by capturing only a partial amount of CO₂ (the amount needed for capture to avoid the carbon tax penalty), as opposed to capturing the entire carbon content of the fuel stream. Also, the use of favorably placed heat exchangers and recycle loops throughout the system improve overall efficiency. Moreover, the disclosed NGCC systems with partial CO2 capture can advantageously be retrofitted to existing NGCC power plants struggling to reduce emissions to avoid potential emissions penalties or carbon taxes. The low temperature operation and small size of the disclosed systems means they can be incorporated into existing plants having minimum real estate without a large capital investment.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A combined cycle system, comprising:
a reformer unit comprising a pre-steam-methane-reformer configured to operate at a temperature of less than about 800 degrees Celsius and to reform a mixed fuel stream to generate a first reformate stream, wherein the mixed fuel stream comprises a first fuel and a steam;
a shift reaction unit comprising a water-gas-shift reactor configured to convert carbon monoxide in the first reformate stream to carbon dioxide and form a second reformate stream;
a carbon dioxide removal unit configured to remove carbon dioxide from the second reformate stream and form a carbon dioxide stream and a third reformate stream; wherein less than about 50 percent of the carbon contained in the first fuel is recovered as carbon dioxide by the carbon removal unit;
a gas turbine unit configured for receiving a mixture of the third reformate stream and a second fuel and generating power and an exhaust gas stream; wherein the exhaust gas stream provides heat to reform the mixed fuel stream; and
a steam generator unit configured to receive the exhaust gas stream, wherein the heat of the exhaust gas stream is transferred to a water stream to generate a cooled exhaust gas stream and the steam for a steam turbine and the mixed fuel stream.

2. The combined cycle system of claim 1, wherein the reformer unit further comprises a heat exchanger configured to receive the first reformate stream and the mixed fuel stream, wherein the heat from the first reformate stream is transferred to the mixed stream to generate a cooled first reformate stream and a heated mixed fuel stream, wherein the heated mixed fuel stream is sent to the pre-steam-methane-reformer.

3. The combined cycle system of claim 1, wherein the shift reaction unit further comprises a heat exchanger configured to receive the second reformate stream and the first fuel, wherein the heat from the second reformate stream is transferred to the first fuel to generate a cooled second reformate stream and a heated first fuel.

4. The combined cycle system of claim 1, further comprising a hydrodesulfurization unit configured to receive the first fuel.

5. The combined cycle system of claim 4, wherein a portion of the third reformate stream is combined with the first fuel and sent to the hydrodesulfurization unit.

6. The combined cycle system of claim 1, wherein the reforming unit has a methane conversion of less than or equal to about 70 percent.

7. The combined cycle system of claim 1, wherein the steam generator unit further comprises a heat recovery steam generator comprising at least two stages, wherein one stage comprises the pre-steam-methane-reformer, and wherein the pre-steam-methane-reformer utilizes heat from the exhaust gas stream to reform the mixed fuel stream.

8. A method for producing power and partially capturing carbon dioxide, comprising:
reforming a mixed fuel stream comprising a first fuel and a steam in a pre-steam-methane-reformer at a temperature of less than about 800 degrees Celsius to produce a first reformate stream comprising hydrogen, carbon monoxide, and steam;
converting the steam and the carbon monoxide in the first reformate stream to a second reformate stream comprising carbon dioxide and hydrogen in a water-gas-shift reactor;
removing the carbon dioxide from the second reformate stream in a carbon dioxide removal unit to produce a carbon dioxide stream and a third reformate stream, wherein less than about 50 percent of the carbon contained in the first fuel is recovered as carbon dioxide by the carbon dioxide removal unit;
combusting a mixture of the third reformate stream and a second fuel stream in a gas turbine unit to generate power and produce an exhaust gas stream; and
utilizing heat in the exhaust gas stream to generate the steam in a heat recovery steam generator, wherein the steam is used to generate power and form the mixed fuel stream with the first fuel.

9. The method of claim 10, wherein the reforming also takes place in the heat recovery steam generator, wherein the heater recovery steam generator has at least two stages, wherein a first stage comprises the pre-steam-methane-reformer, wherein the pre-steam-methane-reformer also utilizes heat from the exhaust gas stream to reform the mixed fuel stream to form the first reformate stream.

10. The method of claim 10, further comprising desulfurizing the first fuel in a hydrodesulfurization unit.

11. The method of claim 10, further comprising transferring heat in a heat exchanger from the first reformate stream to the mixed fuel stream to generate a cooled first reformate stream and a pre-heated mixed fuel stream, wherein the heated mixed fuel stream is sent to the pre-steam-methane-reformer.

12. The method of claim 10, further comprising transferring heat in a heat exchanger from the second reformate stream to the first fuel to generate a cooled second reformate stream and a heated first fuel.

13. A combined cycle system comprising:
a combination unit comprising a heat recovery steam generator, wherein the heat recovery steam generator comprises at least two stages, wherein a first stage comprises a pre-steam-methane-reformer, wherein the pre-steam-methane-reformer is configured to operate at a temperature of less than about 800 degrees Celsius and utilize heat from an hot exhaust gas stream to reform a mixed fuel stream, comprising a first fuel and a steam, to form a first reformate stream, and wherein a second stage utilizes heat from the exhaust gas stream to form the steam;
a shift reaction unit comprising a water-gas-shift reactor configured to convert carbon monoxide in the first reformate stream to carbon dioxide and form a second reformate stream;
a carbon dioxide removal unit configured to remove carbon dioxide from the second reformate stream and form a carbon dioxide stream and a third reformate stream; wherein less than about 50 percent of the carbon contained in the first fuel is recovered as carbon dioxide by the carbon dioxide removal unit; and
a gas turbine unit configured for receiving a second fuel and the third reformate stream and generating power and the exhaust gas stream.

14. The combined cycle system of claim 11, wherein the combination unit further comprises a heat exchanger configured to receive the first reformate stream and the mixed fuel stream, wherein the heat from the first reformate stream is transferred to the mixed stream to generate a cooled first reformate stream and a heated mixed fuel stream, wherein the heated mixed fuel stream is sent to the pre-steam-methane-reformer.

15. The combined cycle system of claim 11, wherein the shift reaction unit further comprises a heat exchanger configured to receive the second reformate stream and the first fuel, wherein the heat from the second reformate stream is transferred to the first fuel to generate a cooled second reformate stream and a heated first fuel.

16. The combined cycle system of claim 11, further comprising a hydrodesulfurization unit configured to receive the first fuel.

17. The combined cycle system of claim 17, wherein a portion of the third reformate stream is combined with the first fuel and sent to the hydrodesulfurization unit.

18. The combined cycle system of claim 11, wherein the reforming unit has a methane conversion of less than or equal to about 70 percent.
